(19) United States
(12) Patent Application Publication
(10) Pub. No.: US 2025/0198685 A1
(43) Pub. Date: Jun. 19, 2025

(54) REFRIGERATOR AND METHOD FOR CONTROLLING THE SAME

(71) Applicant: Samsung Electronics Co., Ltd., Suwon-si (KR)

(72) Inventors: Bokyung LEE, Suwon-si (KR); Jinkook YOON, Suwon-si (KR); Donghyo KIM, Suwon-si (KR); Myungju SHIN, Suwon-si (KR); Sanghoon LEE, Suwon-si (KR); Jisook LEE, Suwon-si (KR)

(21) Appl. No.: 18/967,407

(22) Filed: Dec. 3, 2024

Related U.S. Application Data
(63) Continuation of application No. PCT/KR2024/016643, filed on Oct. 29, 2024.

(30) Foreign Application Priority Data
Dec. 19, 2023 (KR) 10-2023-0186403

(51) Int. Cl. F25D 17/08; A61L 2/00

(52) U.S. Cl. CPC F25D 17/08; A61L 2/0047; F25D 2600/00; F25D 2700/00

(57) ABSTRACT

A refrigerator may include: a storage compartment; a cold air duct; a fan disposed in the cold air duct; a sterilizing lamp configured to sterilize air supplied to the storage compartment through the cold air duct; a contamination sensor for detecting a contamination level in the storage compartment; a first temperature sensor for detecting a first temperature of the sterilizing lamp; a second temperature sensor for detecting a second temperature of the storage compartment; and a controller, configured to: control the fan and the sterilizing lamp; determine a sterilization intensity based on the contamination level; determine a rotation speed of the fan and an input voltage of the sterilizing lamp corresponding to the rotation speed of the fan, based on the sterilization intensity; and adjust the input voltage of the sterilizing lamp based on the first temperature of the sterilizing lamp or the second temperature of the storage compartment.

### CROSS-REFERENCE TO RELATED APPLICATIONS

This application is a continuation of International Application No. PCT/KR2024/016643 designating the United States, filed on Oct. 29, 2024, in the Korean Intellectual Property Receiving Office and claiming priority to Korean Patent Application No. 10-2023-0186403, filed on Dec. 19, 2023, in the Korean Intellectual Property Office, the disclosures of each of which are incorporated by reference herein in their entireties.

### BACKGROUND

### Field

The disclosure relates to a refrigerator and a method for controlling the same.

### Description of Related Art

A refrigerator is an appliance that includes a cabinet (main body) including a storage compartment, and a cold air supply system for supplying cold air to the storage compartment so as to keep food fresh. The storage compartment may include a refrigerating compartment maintained at approximately 0 to 5° C. to store food in a refrigerated manner, and a freezing compartment maintained at approximately 0 to −30° C. to store food in a frozen manner. A door is provided on the front of the main body to open and close the storage compartment.

Various items (e.g., agricultural products, livestock products, and/or fish products, processed products) may be stored in the storage compartment. Such various items may contain bacteria and cause bad odors due to various causes before being placed in the storage compartment or while being stored in the storage compartment. Such bacteria and/or bad odors may remain in the storage compartment. The refrigerator may include a device capable of performing sterilization and/or deodorization of the storage compartment.

### SUMMARY

Embodiments of the disclosure provide a refrigerator and a method for controlling the refrigerator that may control not only a rotation speed of a fan for supplying cold air but also the light irradiation amount from a sterilizing lamp depending on a contamination level of a storage compartment.

Embodiments of the disclosure provide a refrigerator and a method for controlling the refrigerator that may maximize and/or increase sterilization performance by controlling an input voltage of a sterilizing lamp according to a temperature of the sterilizing lamp or a temperature of a storage compartment.

According to an example embodiment of the disclosure, a refrigerator may include: a storage compartment; a cold air duct disposed behind the storage compartment; a fan disposed in the cold air duct; a sterilizing lamp configured to emit ultraviolet light to sterilize air supplied to the storage compartment through the cold air duct; a contamination sensor configured to detect a contamination level in the storage compartment; a first temperature sensor configured to detect a first temperature of the sterilizing lamp; a second temperature sensor configured to detect a second temperature of the storage compartment; and a controller comprising at least one processor, comprising processing circuitry, individually and/or collectively configured to: control the fan and the sterilizing lamp; determine a sterilization intensity based on the contamination level detected by the contamination sensor; determine a rotation speed of the fan and an input voltage of the sterilizing lamp corresponding to the rotation speed of the fan, based on the determined sterilization intensity; and adjust the input voltage of the sterilizing lamp based on the first temperature of the sterilizing lamp or the second temperature of the storage compartment.

According to an example embodiment of the disclosure, a method for controlling a refrigerator may include: detecting a contamination level in a storage compartment by a contamination sensor; determining a sterilization intensity based on the detected contamination level; determining a rotation speed of a fan, configured to move air to the storage compartment, and an input voltage of a sterilizing lamp corresponding to the rotation speed of the fan, based on the determined sterilization intensity, and adjusting the input voltage of the sterilizing lamp based on a first temperature of the sterilizing lamp or a second temperature of the storage compartment.

According to various example embodiments of the disclosure, a refrigerator and a method for controlling the same may control not only a rotation speed of a fan for supplying cold air but also the light irradiation amount from a sterilizing lamp depending on a contamination level of a storage compartment, and thus even in a case where a flow rate of cold air increases due to an increase in the rotation speed of the fan, a sterilization rate may be prevented/reduced from decreasing and a sterilization time may be shortened.

According to various example embodiments of the disclosure, a refrigerator and a method for controlling the same may maximize and/or increase sterilization performance by controlling an input voltage of a sterilizing lamp according to a temperature of the sterilizing lamp or a temperature of a storage compartment.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a perspective view of a refrigerator with an open door according to various embodiments;

FIG. 2 is a side cross-sectional view of a refrigerator according to various embodiments;

FIG. 3 is an enlarged partial cross-sectional view of the sterilizing deodorizing device, the cold air duct, and the fan shown in FIG. 2 according to various embodiments;

FIG. 4 is a perspective view illustrating an example sterilizing deodorizing device mounted in a refrigerating compartment according to various embodiments;

FIG. 5 is an exploded perspective view of a sterilizing deodorizing device according to various embodiments;

FIG. 6 is a block diagram illustrating an example configuration of a refrigerator according to various embodiments;

FIG. 7 is a table illustrating an example of a sterilization intensity corresponding to a contamination level and input voltages of a fan and a sterilizing lamp corresponding to the sterilization intensity according to various embodiments;

FIG. 8 is a table of experimental data showing that the light irradiation amount of a sterilizing lamp varies with temperature;

FIG. 9 is a flowchart illustrating an example method for controlling a refrigerator according to various embodiments;

FIG. 10 is a flowchart illustrating an example method for adjusting an input voltage of the sterilizing lamp of FIG. 9 according to various embodiments;

FIG. 11 is a flowchart illustrating an example method for adjusting an input voltage of the sterilizing lamp of FIG. 9 according to various embodiments; and

FIG. 12 is a flowchart illustrating an example method for controlling a refrigerator to adjust an input voltage of a sterilizing lamp based on a temperature of the sterilizing lamp or a temperature of a storage compartment according to various embodiments.

### DETAILED DESCRIPTION

Various embodiments of the disclosure and terms used herein are not intended to limit the technical features described herein to specific embodiments, and should be understood to include various modifications, equivalents, or substitutions of the corresponding embodiments.

In describing of the drawings, similar reference numerals may be used for similar or related elements.

The singular form of a noun corresponding to an item may include one or more of the items unless clearly indicated otherwise in a related context.

In the disclosure, phrases, such as “A or B”, “at least one of A and B”, “at least one of A or B”, “A, B or C”, “at least one of A, B and C”, and “at least one of A, B, or C” may include any one or all possible combinations of the items listed together in the corresponding phrase among the phrases.

As used herein, the term “and/or” includes any and all combinations of one or more of the associated listed items.

Terms such as “1st”, “2nd”, “primary”, or “secondary” may be used simply to distinguish an element from other elements, without limiting the element in other aspects (e.g., importance or order).

Further, as used in the disclosure, the terms “front”, “rear”, “top”, “bottom”, “side”, “left”, “right”, “upper”, “lower”, and the like are defined with reference to the drawings, and are not intended to limit the shape and position of any element.

It will be understood that when the terms “includes”, “comprises”, “including”, and/or “comprising” are used in the disclosure, they specify the presence of the specified features, figures, steps, operations, components, members, or combinations thereof, but do not preclude the presence or addition of one or more other features, figures, steps, operations, components, members, or combinations thereof.

When a given element is referred to as being “connected to”, “coupled to”, “supported by” or “in contact with” another element, it is to be understood that it may be directly or indirectly connected to, coupled to, supported by, or in contact with the other element. When a given element is indirectly connected to, coupled to, supported by, or in contact with another element, it is to be understood that it may be connected to, coupled to, supported by, or in contact with the other element through a third element.

It will also be understood that when an element is referred to as being “on” another element, it may be directly on the other element or intervening elements may also be present.
A refrigerator 1 according to an embodiment of the disclosure may include a main body (e.g., a cabinet).

The “main body” may include an inner case, an outer case positioned outside the inner case, and an insulation provided between the inner case and the outer case.

The “inner case” may include a case, a plate, a panel, or a liner forming a storage compartment (also referred to as a storage room). The inner case may be formed as one body, or may be formed by assembling a plurality of plates together. The “outer case” may form an appearance of the main body, and be coupled to an outer side of the inner case such that the insulation is positioned between the inner case and the outer case.

The “insulation” may insulate an inside of the storage compartment from an outside of the storage compartment to maintain inside temperature of the storage compartment at appropriate temperature without being influenced by an external environment of the storage compartment. According to an embodiment of the disclosure, the insulation may include a foaming insulation. The foaming insulation may be molded by fixing the inner case and the outer case with jigs, etc. and then injecting and foaming urethane foam as a mixture of polyurethane and a foaming agent between the inner case and the outer case.

According to an embodiment of the disclosure, the insulation may include a vacuum insulation in addition to a foam insulation, or may be configured only with a vacuum insulation instead of a foam insulation. The vacuum insulation may include a core material and a cladding material accommodating the core material and sealing the inside with vacuum or pressure close to vacuum. However, the insulation is not limited to the above-mentioned foam insulation or vacuum insulation, and may include various materials capable of being used for insulation.

The “storage compartment” may include a space defined by the inner case. The storage compartment may further include the inner case defining the space corresponding to the storage compartment. The storage compartment may store a variety of items, such as food, medicines, cosmetics, and the like, and the storage compartment may be configured to be open on at least one side for insertion and removal of the items.

The refrigerator 1 may include one or more storage compartments. In a case in which two or more storage compartments are formed in the refrigerator, the respective storage compartments may have different purposes of use, and may be maintained at different temperatures. To this end, the respective storage compartments may be partitioned by a partition wall including an insulation.

The storage compartment may be maintained within an appropriate temperature range according to a purpose of use, and may include a “refrigerating compartment”, a “freezing compartment”, and a “temperature conversion compartment” according to purposes of use and/or temperature ranges. The refrigerating compartment may be maintained at an appropriate temperature to keep food refrigerating, and the freezing compartment may be maintained at an appropriate temperature to keep food frozen. The “refrigerating” may be keeping food cold without freezing the food, and for example, the refrigerating compartment may be maintained within a range of 0 degrees Celsius to 7 degrees Celsius. The “freezing” may be freezing food or keeping food frozen, and for example, the freezing compartment may be maintained within a range of −20 degrees Celsius to −1 degrees Celsius. The temperature conversion compartment may be used as either a refrigerating compartment or a freezing compartment according to or regardless of a user's selection.

The storage compartment may also be referred to by various terms, such as “vegetable compartment”, “freshness compartment”, “cooling compartment”, and “ice-making compartment”, in addition to “refrigerating compartment”, “freezing compartment”, and “temperature conversion compartment”, and the terms, such as “refrigerating compartment”, “freezing compartment”, “temperature conversion compartment”, etc., as used below are to be understood as representing storage compartments having the corresponding purposes of use and the corresponding temperature ranges.

The refrigerator 1 according to an embodiment of the disclosure may include at least one door configured to open or close the open side of the storage compartment. The respective doors may be provided to open and close one or more storage compartments, or a single door may be provided to open and close a plurality of storage compartments. The door may be rotatably or slidably mounted to the front of the main body.

The “door” may seal the storage compartment in a closed state. The door, like the main body, may include an insulation to insulate the storage compartment in a closed state.

According to an embodiment, the door may include an outer door plate forming the front surface of the door, an inner door plate forming the rear surface of the door and facing the storage compartment, an upper cap, a lower cap, and a door insulation provided therein.

A gasket may be provided on the edge of the inner door plate to seal the storage compartment by coming into close contact with the front surface of the main body when the door is closed. The inner door plate may include a dyke that protrudes rearward to allow a door basket for storing items to be fitted.

According to an embodiment, the door may include a door body and a front panel that is detachably coupled to the front of the door body and forming the front surface of the door. The door body may include an outer door plate forming the front surface of the door body, an inner door plate forming the rear surface of the door body and facing the storage compartment, an upper cap, a lower cap, and a door insulator provided therein.

The refrigerator 1 may be classified as French Door Type, Side-by-side Type, Bottom Mounted Freezer (BMF), Top Mounted Freezer (TMF), or Single Door Refrigerator according to the arrangement of the doors and the storage compartments.

The refrigerator 1 according to an embodiment of the disclosure may include a cold air supply device for supplying cold air to the storage compartment.

The “cold air supply device” may include a machine, an apparatus, an electronic device, and/or a combination system thereof, capable of generating cold air and guiding the cold air to cool the storage compartment.

According to an embodiment of the disclosure, the cold air supply device may generate cold air through a cooling cycle including compression, condensation, expansion, and evaporation processes of refrigerants. To this end, the cold air supply device may include a refrigeration cycle device having a compressor, a condenser, an expander, and an evaporator to drive the refrigeration cycle. According to an embodiment of the disclosure, the cold air supply device may include a semiconductor, such as a thermoelectric element. The thermoelectric element may cool the storage compartment by heating and cooling actions through the Peltier effect.

The refrigerator 1 according to an embodiment of the disclosure may include a machine compartment in which at least some components belonging to the cold air supply device are installed.

The “machine compartment” may be partitioned and insulated from the storage compartment to prevent and/or reduce heat generated by the components installed in the machine compartment from being transferred to the storage compartment. To dissipate heat from the components installed in the machine compartment, the machine compartment may communicate with outside of the main body.

The refrigerator 1 according to an embodiment of the disclosure may include a dispenser provided on the door to provide water and/or ice. The dispenser may be provided on the door to allow access by the user without opening the door.

The refrigerator 1 according to an embodiment of the disclosure may include an ice-making device that produces ice. The ice-making device may include an ice-making tray that stores water, an ice-moving device that separates ice from the ice-making tray, and an ice-bucket that stores ice produced in the ice-making tray.

The refrigerator 1 according to an embodiment of the disclosure may include a controller for controlling the refrigerator 1. The refrigerator 1 may include at least one controller and may include at least one processor. The controller may generate a control signal for controlling the operation of the cold air supply device. For example, the controller may receive temperature information of the storage compartment from a temperature sensor and generate a cooling control signal for controlling the operation of the cold air supply device based on the temperature information of the storage compartment.

Hereinafter, various example embodiments of the disclosure will be described in greater detail with reference to the appended drawings.

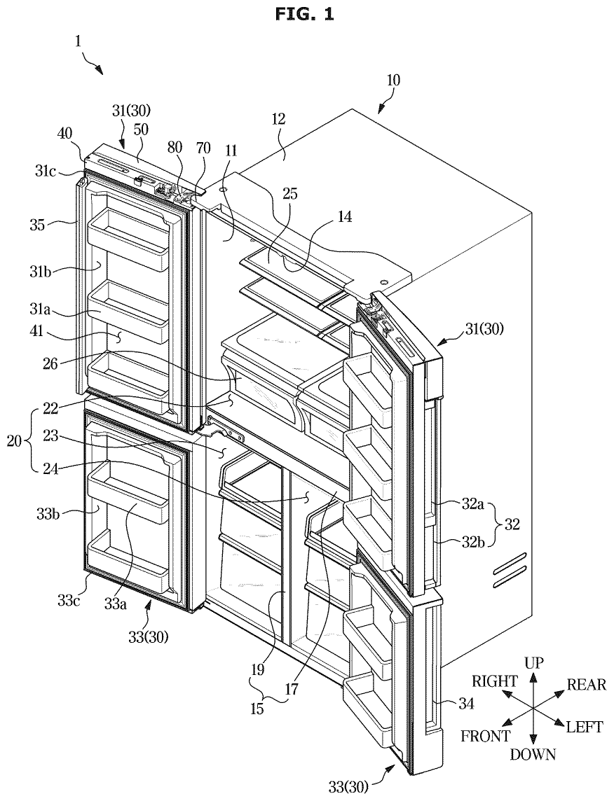

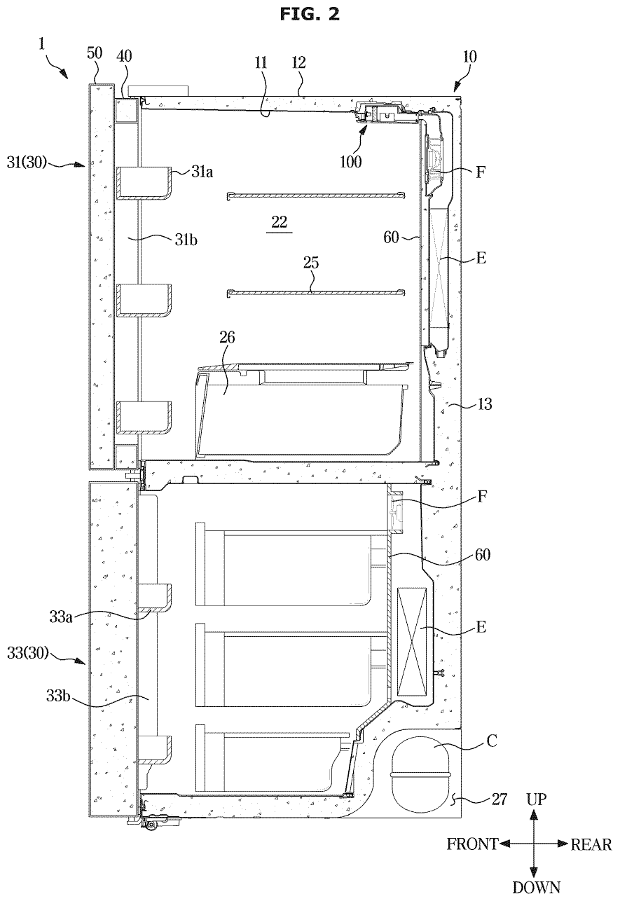

FIG. 1 is a perspective view of a refrigerator with an open door according to various embodiments. FIG. 2 is a side cross-sectional view of a refrigerator according to various embodiments.

Referring to FIG. 1 and FIG. 2, the refrigerator 1 may include a main body (cabinet) 10, a storage compartment 20 formed by being divided vertically inside the main body 10, and a door 30 for opening and closing the storage compartment 20. In addition, the refrigerator 1 may include a cold air supply device for supplying cold air to the storage compartment 20.

The main body 10 may include an inner case 11 forming the storage compartment 20, an outer case 12 coupled to the outer side of the inner case 11 to form an outer appearance, and an insulation 13 foamed between the inner case 11 and the outer case 12 to insulate the storage compartment 20.

A machine compartment 27 may be formed at a lower rear side of the main body 10. The cold air supply device may be disposed in the machine compartment 27. The cold air supply device may include a compressor C for compressing refrigerant, a condenser for condensing the refrigerant, an expansion valve for expanding the refrigerant condensed by the condenser, an evaporator E installed at the rear of the storage compartment 20 to cool the surrounding air, a fan F that moves the air cooled by the evaporator E to the storage compartment 20, and a cold air duct 60 that guides the cold air flowing according to an operation of the fan F to the storage compartment 20. The cold air duct 60 may be disposed at the rear of the storage compartment 20.

The storage compartment 20 may include a refrigerating compartment 22 and freezing compartments 23 and 24. The evaporator E, the fan F, and the cold air duct 60 may be disposed at the rear of each of the refrigerating compartment 22 and the freezing compartments 23 and 24.

The storage compartment 20 may be divided into a plurality of sections by partitions 15. A plurality of shelves 25 and storage containers 26 may be provided in the storage compartment 20 to store food, and the like.

The storage compartment 20 may be divided into a plurality of storage compartments 22, 23, and 24 by the partitions 15. The partitions 15 may include a first partition 17 and a second partition 19. The first partition 17 may horizontally divide the storage compartment 20 into an upper storage compartment 22 and lower storage compartments 23 and 24 in the storage compartment 20. The second partition 19 may vertically divide the lower storage compartments 23 and 24 into the first storage compartment 23 and the second storage compartment 24.

The partition 15 may have a T-shape when the first partition 17 and the second partition 19 are combined. The partition 15 may divide the storage compartment 20 into three spaces. Among the upper storage compartment 22 and the lower storage compartments 23 and 24 divided by the first partition 17, the upper storage compartment 22 may be used as a refrigerating compartment. At least one of the two lower storage compartments 23 and 24 may be used as a freezing compartment.

For example, the entire lower storage compartments 23 and 24 may be used as a freezing compartment. The first lower storage compartment 23 may be used as a freezing compartment and the second lower storage compartment 24 may be used as a refrigerating compartment. The first lower storage compartment 23 may be used as a freezing compartment and the second lower storage compartment 24 may be used as a refrigerating compartment. Both the first lower storage compartment 23 and the second lower storage compartment 24 may be used as refrigerating compartments.

The storage compartment 20 is not limited to the above example. The storage compartment 20 may be formed in various ways depending on the design.

The refrigerator 1 may include a door 30. The door 30 may open or close each of the refrigerating compartment 22 and the freezing compartments 23 and 24. The door 30 may be rotatably coupled to the main body 10. The door 30 may include a pair of refrigerating compartment doors 31 and a pair of freezing compartment doors 33. The refrigerating compartment doors 31 may open and close the refrigerating compartment 22. The freezing compartment doors 33 may open and close the freezing compartments 23 and 24.

The pair of refrigerating compartment doors 31 may be provided with a first door handle 32a and a second door handle 32b. A portion of the refrigerating compartment 22 or the entire refrigerating compartment 22 may be opened or closed by at least one of the pair of refrigerating compartment doors 31.

A rotating bar 35 may be provided on at least one of the pair of refrigerating compartment doors 31 to seal the refrigerating compartment doors 31 without a gap formed between the refrigerating compartment doors 31 when the refrigerating compartment doors 31 are closed. The rotating bar 35 may be rotatably coupled to at least one of the pair of refrigerating compartment doors 31. The rotating bar 35 may be rotated by a rotating guide 14 formed on the main body 10 according to the opening and closing of the refrigerating compartment doors 31.

A freezing compartment door handle 34 may be provided on each of the pair of freezing compartment doors 33. The freezing compartment doors 33 may be provided as a sliding door.

Door shelves 31a and 33a for storing food may be arranged on the back side of the refrigerating compartment doors 31 and the freezing compartment doors 33.

Shelf supports 31b and 33b for supporting the left and right sides of the door shelves 31a and 33a may be arranged on each of the refrigerating compartment doors 31 and the freezing compartment doors 33. The shelf supports 31b and 33b may be detachable from each of the doors 31 and 33.

In addition, first gaskets 31c and 33c may be provided on the rear edge of each of the doors 31 and 33 to seal the gap between the doors 31 and 33 and the main body 10 in a state where the doors are closed. The first gaskets 31c and 33c may be installed in a loop shape along the rear edge of each of the doors 31 and 33, and may include a magnet inside.

The refrigerating compartment doors 31 may be provided as a double door including a first door 40 and a second door 50. The first door 40 may be rotatably coupled to the main body 10 by a first hinge 70 and may open and close the refrigerating compartment 22. The door shelf 31a, the shelf support 31b, and the first gasket 31c described above may be provided on the first door 40.

The first door 40 may include an opening 41. A user may store food in the door shelf 31a or take food out from the door shelf 31a through the opening 41 in a state where the first door 40 is closed. The opening 41 passes through the first door 40 and may be opened and closed by the second door 50.

The second door 50 may be provided on the front of the first door 40 so as to open and close the opening 41 of the first door 40. The second door 50 may be rotatable in the same direction as the first door 40. For example, the second door 50 may be rotatably supported by a second hinge 80 installed on the first door 40. The second hinge 80 may also be installed on the main body 10.

The second door 50 may include a second gasket for maintaining airtightness with the first door 40. The second gasket may be installed in a loop shape along the rear edge of the second door 50, and may include a magnet inside.

A sterilizing deodorizing device 100 for sterilizing and deodorizing air may be provided in the storage compartment 20. The sterilizing deodorizing device 100 may be disposed in the refrigerating compartment 22. The sterilizing deodorizing device 100 may be installed on an upper wall of the refrigerating compartment 22. The sterilizing deodorizing device 100 may also be referred to as a sterilization device (sterilizer).

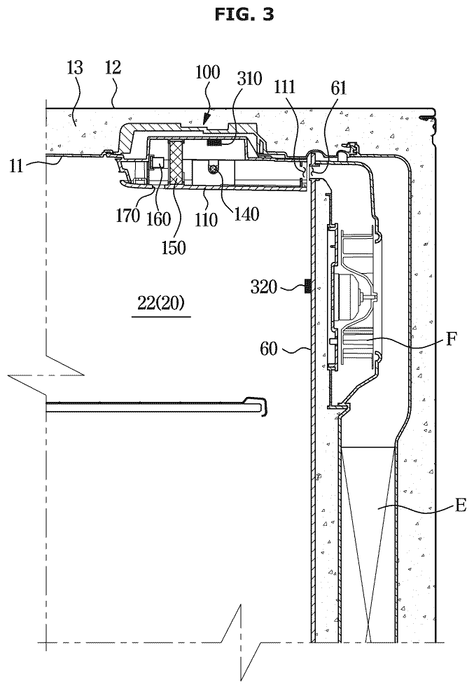

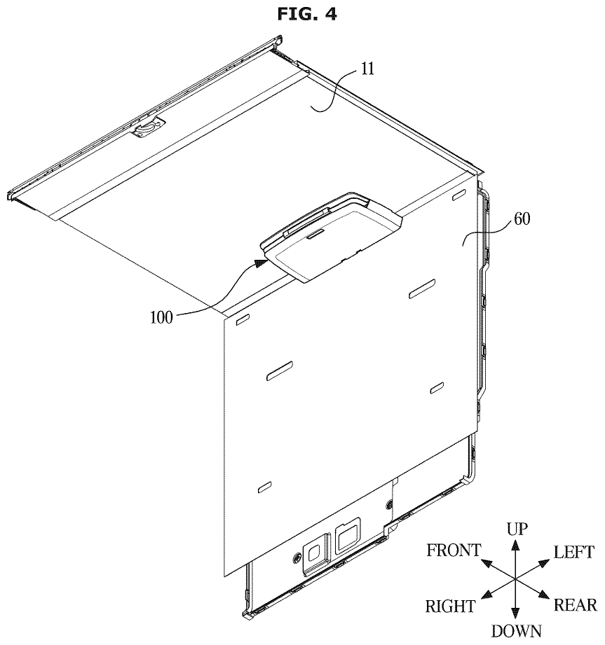

FIG. 3 is an enlarged cross sectional view of a sterilizing deodorizing device, a cold air duct, and a fan shown in FIG. 2 according to various embodiments. FIG. 4 is a perspective view illustrating a sterilizing deodorizing device mounted in a refrigerating compartment according to various embodiments.

Referring to FIG. 3 and FIG. 4, the sterilizing deodorizing device 100 may be mounted on an upper wall of the refrigerating compartment 22. The upper wall of the refrigerating compartment 22 may correspond to the inner case 11 of the storage compartment 20 described above. The sterilizing deodorizing device 100 may include a housing 110, and the housing 110 may be mounted on the upper wall of the refrigerating compartment 22. The location of the sterilizing deodorizing device 100 is not limited to the refrigerating compartment 22. The sterilizing deodorizing device 100 may be installed in a location other than the refrigerating compartment 22. The sterilizing deodorizing device 100 may be installed in the freezing compartments 23 and 24.

The sterilizing deodorizing device 100 may be connected to the cold air duct 60. The cold air duct 60 may be disposed on the rear side of the refrigerating compartment 22. The air cooled by the evaporator E may move through the cold air duct 60 according to an operation of the fan F. The cold air moving through the cold air duct 60 may be discharged from the cold air duct 60 through a cold air outlet 61. The cold air discharged from the cold air outlet 61 may be introduced into the sterilizing deodorizing device 100. The cold air outlet 61 may be connected to an inlet 111 of the sterilizing deodorizing device 100. The cold air discharged from the cold air outlet 61 may be introduced into the sterilizing deodorizing device 100 through the inlet 111.

The sterilizing deodorizing device 100 may include a sterilizing lamp 140. The sterilizing lamp 140 may sterilize the air introduced into the sterilizing deodorizing device 100. The sterilizing lamp 140 may emit ultraviolet light for sterilizing air. For example, the sterilizing lamp 140 may be provided as an ultraviolet C (UVC) lamp.

The sterilizing deodorizing device 100 may include a deodorizing filter 150. The deodorizing filter 150 may remove odors from the air introduced into the sterilizing deodorizing device 100. The deodorizing filter 150 may be activated by ultraviolet light emitted from the sterilizing lamp 140. After passing through the sterilizing lamp 140 and the deodorizing filter 150, the air may be discharged into the refrigerating compartment 22 through an outlet 170.

In addition, the sterilizing deodorizing device 100 may include a deodorizing lamp 160. The deodorizing lamp 160 may emit ultraviolet light to activate the deodorizing filter 150. For example, the deodorizing lamp 160 may be provided as an ultraviolet A (UVA) lamp. The deodorizing lamp 160 may be positioned adjacent to the outlet 170. The air introduced into the housing 110 may pass through the sterilizing lamp 140, the deodorizing filter 150, and the deodorizing lamp 160, and then be discharged through the outlet 170.

The refrigerator 1 may include a temperature sensor. For example, the temperature sensor may include a first temperature sensor 310 detecting a first temperature of the sterilizing lamp 140 and a second temperature sensor 320 detecting a second temperature of the storage compartment 20. The first temperature sensor 310 may be positioned in the housing 110 of the sterilizing deodorizing device 100. The second temperature sensor 320 may be positioned in the storage compartment 20.

The location of the first temperature sensor 310 is not limited to the position shown. The first temperature sensor 310 may be installed in various locations capable of measuring the first temperature of the sterilizing lamp 140. For example, the first temperature sensor 310 may be embedded in the sterilizing lamp 140. The location of the second temperature sensor 320 is also not limited to the position shown. The second temperature sensor 320 may be installed in various locations capable of measuring the second temperature of the storage compartment 20.

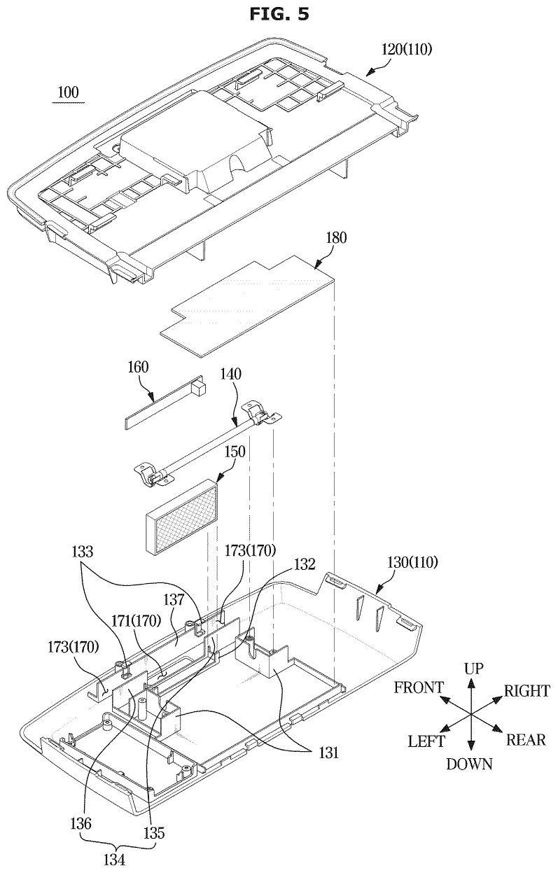

FIG. 5 is an exploded perspective view of a sterilizing deodorizing device according to various embodiments.

Referring to FIG. 5, the sterilizing deodorizing device 100 may include the housing 110. The housing 110 may include a first housing 120 mounted on the upper wall of the refrigerating compartment 22 and a second housing 130 mounted on a lower part of the first housing 120.

The second housing 130 may include a sterilizing lamp mounting portion 131 on which the sterilizing lamp 140 is mounted. The sterilizing lamp mounting portion 131 may be disposed on the lower wall of the second housing 130. The sterilizing lamp mounting portion 131 may be disposed on the central portion of the lower wall of the second housing 130. Both ends of the sterilizing lamp 140 having a cylindrical shape may be mounted on the sterilizing lamp mounting portion 131.

The second housing 130 may include a deodorizing filter mounting portion 132 on which the deodorizing filter 150 is mounted. The deodorizing filter mounting portion 132 may be provided on the lower wall of the second housing 130. The deodorizing filter mounting portion 132 may be disposed in front of the sterilizing lamp mounting portion 131.

The second housing 130 may include a deodorizing lamp mounting portion 133 on which the deodorizing lamp 160 is mounted. The deodorizing lamp mounting portion 133 may be provided on the lower wall of the second housing 130. The deodorizing lamp mounting portion 133 may be disposed in front of the deodorizing filter mounting portion 132. That is, the deodorizing lamp mounting portion 133 may be disposed on the opposite side of the sterilizing lamp mounting portion 131 based on the deodorizing filter mounting portion 132.

The inlet 111, the sterilizing lamp mounting portion 131, the deodorizing filter mounting portion 132, and the deodorizing lamp mounting portion 133 formed by the housing 110 may be arranged in sequence along a direction of air flow into the inside of the housing 110. In other words, the inlet 111, the sterilizing lamp 140, the deodorizing filter 150, and the deodorizing lamp 160 may be arranged in sequence from the rear to the front of the housing 110.

The second housing 130 may include deodorizing filter ribs 134 provided on the left and right sides of the deodorizing filter 150, respectively. The deodorizing filter ribs 134 may be provided on the left and right sides of the deodorizing filter mounting portion 132, respectively. The deodorizing filter ribs 134 may prevent/reduce ultraviolet light emitted from the sterilizing lamp 140 from leaking out through the outlet 170. In other words, the deodorizing filter ribs 134 may prevent/reduce ultraviolet light emitted from the sterilizing lamp 140 from leaking into the refrigerating compartment 22 through the outlet 170.

The deodorizing filter ribs 134 may include a first deodorizing filter rib 135 provided on the right side of the deodorizing filter 150 and a second deodorizing filter rib 136 provided on the left side of the deodorizing filter 150. The first deodorizing filter rib 135 may be provided on the right side of the deodorizing filter mounting portion 132. The second deodorizing filter rib 136 may be provided on the left side of the deodorizing filter mounting portion 132. A width between a right end of the first deodorizing filter rib 135 and a left end of the second deodorizing filter rib 136 may be longer than a length of the sterilizing lamp 140.

The second housing 130 may include a deodorizing lamp rib 137 provided in front of the deodorizing lamp 160. The deodorizing lamp rib 137 may be provided in front of the deodorizing lamp mounting portion 133. The deodorizing lamp rib 137 may prevent/reduce ultraviolet light emitted from the sterilizing lamp 140 from leaking out through the outlet 170. That is, the deodorizing lamp rib 137 may prevent/reduce ultraviolet light emitted from the sterilizing lamp 140 from leaking into the refrigerating compartment 22 through the outlet 170. In addition, the deodorizing lamp rib 137 may prevent/reduce ultraviolet light emitted from the deodorizing lamp 160 from leaking into the refrigerating compartment 22 through the outlet 170. A length of the deodorizing lamp rib 137 may be longer than a length of the deodorizing filter 150.

The deodorizing filter ribs 134 and the deodorizing lamp rib 137 may guide the air flowing into the housing 110 to the outlet 170. Inside the housing 110, the air may pass through a space between the deodorizing filter ribs 134 and the deodorizing lamp rib 137 and be discharged to the outlet 170.

The sterilizing lamp 140 may have a cylindrical shape. Ultraviolet light may be radiated through a curved surface of the cylindrical sterilizing lamp 140. The sterilizing lamp 140 may be mounted on the sterilizing lamp mounting portion 131 provided in the second housing 130. Both ends of the sterilizing lamp 140 may be mounted on the sterilizing lamp mounting portion 131. The ultraviolet light emitted from the sterilizing lamp 140 may sterilize the air flowing into the housing 110. The sterilized air may be discharged into the refrigerating compartment 22 through the outlet 170.

The sterilizing lamp 140 may be positioned between the inlet 111 and the deodorizing filter 150. The air sterilized by the ultraviolet light of the sterilizing lamp 140 may be introduced into the deodorizing filter 150. Because the sterilizing lamp 140 is positioned in the central portion of the housing 110, an area where the ultraviolet light reach inside the housing 110 may be maximized and/or increased.

The deodorizing filter 150 may be activated by the ultraviolet light emitted from the sterilizing lamp 140. In a case where the deodorizing filter 150 is activated by the ultraviolet light emitted from the sterilizing lamp 140, the sterilized air may be deodorized by the deodorizing filter 150, and then be discharged into the refrigerating compartment 22 through the outlet 170. Sterilization and/or deodorization of air may be performed by a single sterilizing lamp 140.

The sterilizing deodorizing device 100 may include the deodorizing filter 150. The deodorizing filter 150 may be mounted on the deodorizing filter mounting portion 132 in the second housing 130. The deodorizing filter 150 may be disposed in front of the sterilizing lamp 140. The sterilizing lamp 140 may be positioned between the inlet 111 and the deodorizing filter 150. The deodorizing filter 150 may be activated by ultraviolet light emitted from the sterilizing lamp 140. The activated deodorizing filter 150 may deodorize the air introduced through the inlet 111.

The sterilizing deodorizing device 100 may include the deodorizing lamp 160. The deodorizing lamp 160 may be mounted on the deodorizing lamp mounting portion 133 in the second housing 130. The deodorizing lamp 160 may be disposed in front of the deodorizing filter 150. That is, the deodorizing lamp 160 may be provided on the opposite side of the sterilizing lamp 140 based on the deodorizing filter 150.

The sterilizing lamp 140 and the deodorizing lamp 160 may selectively activate the deodorizing filter 150. In a case where at least one of the sterilizing lamp 140 or the deodorizing lamp 160 is turned on, the deodorizing filter 150 may be activated, and in a case where both the sterilizing lamp 140 and the deodorizing lamp 160 are turned off, the deodorizing filter 150 may be deactivated.

Operation of the sterilizing lamp 140 may cause the air supplied to the storage compartment 20 to be sterilized and deodorized simultaneously. In a case where only the deodorizing lamp 160 operates without operation of the sterilizing lamp 140, deodorization of the air supplied to the storage compartment 20 may be performed.

In a case where sterilization and deodorization of the air supplied to the storage compartment 20 are required, the refrigerator 1 may operate the sterilizing lamp 140. For example, in a case where the door of the storage compartment 20 is opened and then closed, it may be determined that sterilization operation for sterilizing the air is required. In addition, sterilization operation may be performed at defined (e.g., predetermined) time intervals.

The outlet 170 for discharging the sterilized and deodorized air may be provided in the second housing 130. The outlet 170 may be formed between the deodorizing filter 150 and the deodorizing lamp 160. The outlet 170 may include a first outlet 171 formed between the deodorizing filter 150 and the deodorizing lamp 160. In addition, the outlet 170 may include second outlets 173 formed on the left and right sides of the first outlet 171 by the deodorizing filter ribs 134 and the deodorizing lamp rib 137. The sterilized and deodorized air may be discharged into the storage compartment 20 through the first outlet 171 and the second outlets 173.

From the rear to the front of the housing 110, the inlet 111, the sterilizing lamp 140, the deodorizing filter 150, the outlet 170, and the deodorizing lamp 160 may be arranged in sequence. Because the inlet 111, the sterilizing lamp 140, the deodorizing filter 150, the outlet 170, and the deodorizing lamp 160 are arranged in a straight line, air flow loss may be minimized and/or reduced while sterilizing and/or deodorizing air is performed.

The sterilizing deodorizing device 100 may include a heat-radiating tape 180 attached to an inner side of the housing 110. The heat-radiating tape 180 may be attached to an inner side of the second housing 130. The heat-radiating tape 180 may prevent and/or reduce heat generated from the sterilizing lamp 140 from being released to the outside.

The heat-radiating tape 180 may prevent and/or reduce heat generated from the sterilizing lamp 140 from moving to the storage compartment 20. An influence of the heat generation from the sterilizing lamp 140 on the temperature of the storage compartment 20 may be minimized and/or reduced by the heat-radiating tape 180.

In addition, the heat-radiating tape 180 may include a reflective tape made of a metal material to reflect ultraviolet light. The ultraviolet light emitted from the sterilizing lamp 140 may be reflected by the heat-radiating tape 180. Accordingly, the ultraviolet light may be prevented/reduced from directly reaching the surface of the housing 110, and light degradation of the housing 110 may be prevented and/or reduced. The ultraviolet light reflected by the heat-radiating tape 180 may sterilize the air again.

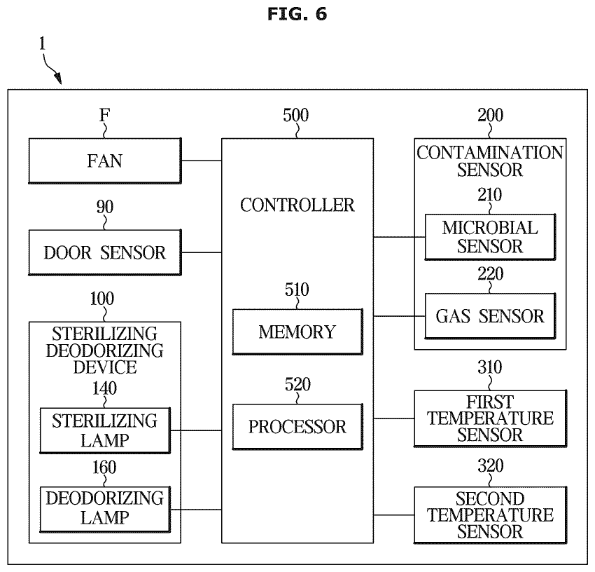

FIG. 6 is a block diagram illustrating an example configuration of a refrigerator according to various embodiments.

Referring to FIG. 6, the refrigerator 1 may include a fan F, a door sensor 90, the sterilizing deodorizing device (e.g., including a lamp) 100, a contamination sensor 200, the first temperature sensor 310, the second temperature sensor 320, and the controller (e.g., including circuitry) 500. The controller 500 may be electrically connected to various electronic devices and/or electronic components of the refrigerator 1, and may control the electronic devices and/or electronic components. The controller 500 may control the overall operation of the refrigerator 1.

The components of the refrigerator 1 electrically connected to the controller 500 are not limited to those illustrated in FIG. 6. The refrigerator 1 may further include other components in addition to the components described in FIG. 6. For example, the refrigerator 1 may include a user interface and a communication interface. In addition, the controller 500 may control the aforementioned cold air supply device.

The processor 520 may include various processing circuitry and process an input (e.g., a user input) of the user interface according to the program and/or data recorded/stored in a memory 510, and control the operation of the user interface. The user interface may include an input interface and an output interface. The processor 520 may receive a user input from the user interface. In addition, the processor 520 may transmit a display signal and image data for displaying images on the user interface in response to the user input to the user interface.

The input interface may include various input devices capable of obtaining a user input, such as keys, buttons, a touch screen, and a microphone. The input interface may transmit an electrical signal corresponding to the user input to the processor 520. The output interface may include a display that outputs visual elements and a speaker that outputs sounds. The output interface may output various notifications, messages, information, etc. generated by the processor 520.

The refrigerator 1 may include the communication interface including various communication circuitry for communicating with an external device. The communication interface may communicate with an external device such as a server, a mobile device, and other home appliances through a nearby access point (AP). The access point (AP) may connect a local area network (LAN) to which the refrigerator 1 or the user device is connected to a wide area network (WAN) to which the server is connected. The refrigerator 1 or the user device may be connected to the server through the wide area network (WAN).

The controller 500 may include the memory 510 that stores or records a program and/or data for controlling the refrigerator 1, and the processor (e.g., including processing circuitry) 520 that generates a control signal for controlling various electronic devices and/or electronic components provided in the refrigerator 1 according to the program and/or data stored in the memory 510. The controller 500 may include at least one processor 520 and at least one memory 510.

At least one controller 500 may be provided. For example, a plurality of controllers or one integrated controller may be provided to individually control various electronic devices and/or electronic components of the refrigerator 1.

The memory 510 may store or record various information, data, instructions, and programs required for the operation of the refrigerator 1. The memory 510 may store temporary data generated during the generation of a control signal for controlling the components included in the refrigerator 1. The memory 510 may include at least one of a volatile memory and a non-volatile memory, or a combination thereof.

The processor 520 and the memory 510 may be provided integrally or separately. At least one processor 520 may be provided. For example, the processor 520 may include a main processor and at least one sub-processor. The processor 520 may include a separate neural network processing unit (NPU) that performs an artificial intelligence (AI) model operation. Various electronic devices and/or electronic components of the refrigerator 1 may be controlled by separate processors or by one integrated processor. At least one memory 510 may be provided. The processor 520 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The fan F causes air to flow. The fan F may move air cooled by the evaporator E to the storage compartment 20. According to an operation of the fan F, the air may move to the sterilizing deodorizing device 100 through the cold air duct 60. The controller 500 may adjust a rotation speed of the fan F. The controller 500 may adjust an input voltage of the fan F to adjust the rotation speed of the fan F. An increase in the input voltage of the fan F may increase the rotation speed of the fan F. The increase in the rotation speed of the fan F may increase an air flow rate into the sterilizing deodorizing device 100 and the storage compartment 20. Conversely, a decrease in the rotation speed of the fan F may decrease the air flow rate into the sterilizing deodorizing device 100 and the storage compartment 20.

The door sensor 90 may detect the opening and/or closing of the door 30. The door sensor 90 may be disposed on each of the plurality of doors. The door sensor 90 may transmit an opening signal corresponding to the opening of the door 30 to the controller 500. The door sensor 90 may transmit a closing signal corresponding to the closing of the door 30 to the controller 500. The controller 500 may identify the opening or closing of the door 30 based on the signal transmitted from the door 30.

The sterilizing deodorizing device 100 may include the sterilizing lamp 140. In addition, the sterilizing deodorizing device 100 may include the deodorizing lamp 160. The sterilizing lamp 140 may emit ultraviolet light to sterilize air supplied to the storage compartment 20 through the cold air duct 60. For example, the sterilizing lamp 140 may be provided as an ultraviolet C (UVC) lamp. The deodorizing lamp 160 may emit ultraviolet light to activate the deodorizing filter 150. For example, the deodorizing lamp 160 may be provided as an ultraviolet A (UVA) lamp. The controller 500 may control the sterilizing lamp 140 and the deodorizing lamp 160.

Because the deodorizing filter 150 may also be activated by ultraviolet light emitted from the sterilizing lamp 140, the deodorizing lamp 160 may be omitted depending on the design.

The controller 500 may adjust an input voltage of the sterilizing lamp 140. An increase in the input voltage of the sterilizing lamp 140 may increase the light irradiation amount of the sterilizing lamp 140. In other words, the increase in the input voltage of the sterilizing lamp 140 may increase the intensity of ultraviolet light emitted from the sterilizing lamp 140. Conversely, a decrease in the input voltage of the sterilizing lamp 140 may decrease the intensity of ultraviolet light emitted from the sterilizing lamp 140.

The increase in the rotation speed of the fan F may increase a flow rate of cold air passing through the sterilizing lamp 140. The increase in the flow rate of cold air may cause the time during which the cold air is exposed to ultraviolet light emitted from the sterilizing lamp 140 to become shorter. In a case where there is no change in the input voltage of the sterilizing lamp 140 despite the increased flow rate of the cold air, a sterilizing rate, e.g., a sterilization performance, may decrease.

The controller 500 may determine the input voltage of the sterilizing lamp 140 to correspond to the rotation speed of the fan F or the input voltage of the fan F. The input voltage of the fan F may be referred to as a first input voltage. The input voltage of the sterilizing lamp 140 may be referred to as a second input voltage. The controller 500 may increase the input voltage of the sterilizing lamp 140 based on the increase in the rotation speed of the fan F. The controller 500 may decrease the input voltage of the sterilizing lamp 140 based on the decrease in the rotation speed of the fan F.

The contamination sensor 200 may detect a contamination level in the storage compartment 20. The contamination sensor 200 may be disposed in the storage compartment 20. For example, the contamination sensor 200 may include at least one of a microbial sensor 210 and/or a gas sensor 220. The microbial sensor 210 may detect a microbial concentration in the storage compartment 20. The gas sensor 220 may detect a gas concentration in the storage compartment 20. The contamination level in the storage compartment 20 may correspond to at least one of the microbial concentration or the gas concentration. The contamination sensor 200 may transmit information about the contamination level corresponding to at least one of the microbial concentration or the gas concentration to the controller 500.

The microbial sensor 210 may detect various types of microorganisms. For example, the microbial sensor 210 may detect a concentration of microorganisms, such as mold, bacteria, yeast, and viruses. The microbial sensor 210 may transmit an electrical signal corresponding to the microbial concentration to the controller 500. The controller 500 may identify the microbial concentration in the storage compartment 20 based on the electrical signal received from the microbial sensor 210.

The gas sensor 220 may detect various types of gases. For example, the gas sensor 220 may detect a concentration of gases, such as acetic acid, aldehyde, sulfur compounds, alcohol, ammonia, volatile organic acids, and methane gas, generated from various foods placed in the storage compartment 20. The gas sensor 220 may transmit an electrical signal corresponding to the gas concentration to the controller 500. The controller 500 may identify the gas concentration in the storage compartment 20 based on the electrical signal received from the gas sensor 220.

The controller 500 may determine the contamination level in the storage compartment 20 based on at least one of the microbial concentration or the gas concentration. Data about the contamination level corresponding to at least one of the microbial concentration or the gas concentration may be stored in the memory 510. The controller 500 may read the data stored in the memory 510 to determine the contamination level in the storage compartment 20.

The contamination sensor 200 is not limited to the above examples. In addition to the microbial sensor 210 and the gas sensor 220, a sensor capable of detecting an environment in the storage compartment 20 may be included in the contamination sensor 200.

The controller 500 may start a sterilization operation based on the door 30 of the storage compartment 20 being opened and then closed. That is, when the door 30 is opened and then closed, the air in the storage compartment 20 is required to be sterilized. In addition, the controller 500 may perform the sterilization operation at defined (predetermined) time intervals. The controller 500 may operate the fan F and the sterilizing lamp 140 for the sterilization operation.

The controller 500 may determine a sterilization intensity to perform the sterilization operation. The controller 500 may determine the sterilization intensity for sterilizing the inside of the storage compartment 20 based on the contamination level in the storage compartment 20. For example, the controller 500 may determine the sterilization intensity to be stronger as the contamination level in the storage compartment 20 is higher. The controller 500 may determine the sterilization intensity to be stronger as the microbial concentration in the storage compartment 20 is higher. The controller 500 may determine the sterilization intensity to be stronger as the gas concentration in the storage compartment 20 is higher. The sterilization intensity may be determined to be low, medium, or high. The terms indicating the sterilization intensity are relative and may be expressed in different terms depending on the design.

The controller 500 may determine a rotation speed of the fan F based on the sterilization intensity. The controller 500 may determine the input voltage of the sterilizing lamp 140 corresponding to the rotation speed of the fan F. For example, the higher the sterilization intensity is determined, the higher the rotation speed of the fan F may be determined. As the rotation speed of the fan F increases, the flow rate of the air supplied to the storage compartment 20 may increase, and thus the contamination level of the storage compartment 20 may be reduced quickly. The higher the sterilization intensity is determined, the higher the input voltage of the sterilizing lamp 140 may also be determined. As the input voltage of the sterilizing lamp 140 increases, the intensity of ultraviolet light emitted from the sterilizing lamp 140 may increase, and the sterilization rate of the air passing through the sterilizing lamp 140 may increase.

The first temperature sensor 310 may detect a temperature of the sterilizing lamp 140. The first temperature sensor 310 may be located adjacent to the sterilizing lamp 140. For example, the first temperature sensor 310 may be disposed in the housing 110 of the sterilizing deodorizing device 100. The first temperature sensor 310 may be embedded in the sterilizing lamp 140. The first temperature sensor 310 may transmit an electrical signal corresponding to the temperature of the sterilizing lamp 140 to the controller 500. The controller 500 may identify the temperature of the sterilizing lamp 140 based on the electrical signal received from the first temperature sensor 310.

The second temperature sensor 320 may detect a temperature of the storage compartment 20. The second temperature sensor 320 may be disposed in the storage compartment 20. The second temperature sensor 320 may transmit an electrical signal corresponding to the temperature of the storage compartment 20 to the controller 500. The controller 500 may identify the temperature of the storage compartment 20 based on the electrical signal received from the second temperature sensor 320.

The sterilizing lamp 140 may emit heat. The higher the input voltage of the sterilizing lamp 140, the more heat the sterilizing lamp 140 may emit. The heat generated by the sterilizing lamp 140 may increase the temperature of the sterilizing lamp 140.

The temperature of the storage compartment 20 may affect the temperature of the sterilizing lamp 140. The cold air discharged from the cold air duct 60 may pass through the sterilizing lamp 140 and then be supplied to the storage compartment 20. The cold air supplied to the storage compartment 20 may have a greater effect on the temperature change of the sterilizing lamp 140 than the heat generated by the sterilizing lamp 140.

The lower the temperature of the cold air supplied to the storage compartment 20, the lower the temperature of the sterilizing lamp 140 may be detected. In other words, the lower the temperature of the storage compartment 20, the lower the temperature of the sterilizing lamp 140 may be detected. The temperature of the sterilizing lamp 140 may be referred to as a first temperature. The temperature of the storage compartment 20 may be referred to as a second temperature.

The temperature of the sterilizing lamp 140 may affect a sterilization performance of the sterilizing lamp 140. As the temperature of the sterilizing lamp 140 increases, the light irradiation amount may be reduced and the sterilization performance may deteriorate. As the temperature of the sterilizing lamp 140 is relatively lower, the sterilization performance may be improved. Even in a case where the input voltage of the sterilizing lamp 140 is maintained the same, the light irradiation amount may be detected differently depending on the temperature of the sterilizing lamp 140.

For example, the sterilization performance of the sterilizing lamp 140 is related not only to the input voltage of the sterilizing lamp 140 but also to the temperature of the sterilizing lamp 140. In a case where the temperature of the sterilizing lamp 140 exceeds a limit temperature, the sterilizing lamp 140 may fail. In order to maximize and/or increase/improve the performance of the sterilizing lamp 140, the controller 500 may adjust the input voltage of the sterilizing lamp 140 according to the temperature of the sterilizing lamp 140 or the temperature of the storage compartment 20.

The controller 500 may increase the input voltage of the sterilizing lamp 140 based on the first temperature of the sterilizing lamp 140 being lower than a defined (predetermined) limit temperature. The controller 500 may decrease the input voltage of the sterilizing lamp 140 based on the first temperature of the sterilizing lamp 140 reaching the defined (e.g., predetermined) limit temperature.

The controller 500 may increase the input voltage of the sterilizing lamp 140 based on the second temperature of the storage compartment 20 being lower than a defined (predetermined) threshold temperature. The controller 500 may decrease the input voltage of the sterilizing lamp 140 in a case where the second temperature of the storage compartment 20 is higher than or equal to the defined (e.g., predetermined) threshold temperature.

The controller 500 may turn off the sterilizing lamp 140 or apply a defined (e.g., predetermined) default voltage to the sterilizing lamp 140, based on a cumulative sterilization time reaching a defined (e.g., predetermined) threshold time. The controller 500 may count the cumulative sterilization time from the time the sterilization operation starts.

In a case where the refrigerator 1 includes the deodorizing lamp 160, the deodorizing filter 150 may be activated by the deodorizing lamp 160, and thus the controller 500 may stop applying voltage to the sterilizing lamp 140 and turn off the sterilizing lamp 140 in a case where the cumulative sterilization time reaches the threshold time. In a case where the refrigerator 1 does not include the deodorizing lamp 160, the controller 500 may apply a default voltage to the sterilizing lamp 140 to maintain the activation state of the deodorizing filter 150. The default voltage may correspond to a minimum voltage required to activate the deodorizing filter 150. That is, a deodorization operation may continue even in a case where the sterilization operation is completed.

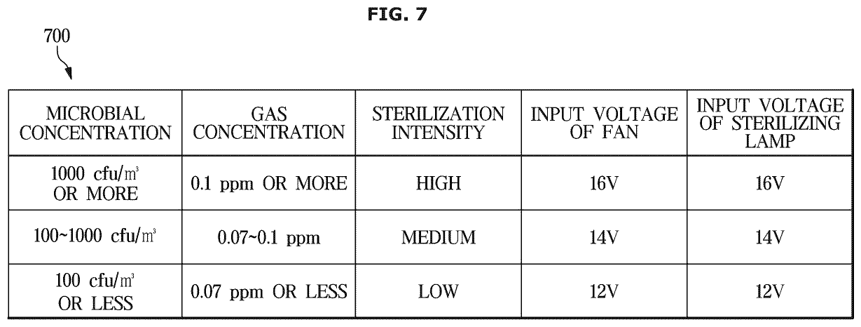

FIG. 7 is a table illustrating an example of a sterilization intensity corresponding to a contamination level and input voltages of a fan and a sterilizing lamp corresponding to the sterilization intensity according to various embodiments.

Referring to FIG. 7, illustrates data representing a relationship between a microbial concentration and a gas concentration in the storage compartment 20 detected by the contamination sensor 200, a sterilization intensity, an input voltage of the fan F, and an input voltage of the sterilizing lamp 140.

The controller 500 may determine the sterilization intensity based on at least one of the microbial concentration or gas concentration in the storage compartment 20. For example, in a case where the microbial concentration is 100 cfu/m³ or less and/or the gas concentration is 0.07 ppm or less, the sterilization intensity may be determined as ‘low’. In a case where the microbial concentration is greater than or equal to 100 cfu/m³ and less than 1000 cfu/m³ and/or the gas concentration is greater than or equal to 0.07 ppm and less than 0.1 ppm, the sterilization intensity may be determined as ‘medium’. In a case where the microbial concentration is greater than or equal to 1000 cfu/m³ and/or the gas concentration is greater than or equal to 0.1 ppm, the sterilization intensity may be determined as ‘high’. The terms about the sterilization intensity, e.g., low, medium, and high, are relative terms, and the sterilization intensity may be expressed in different terms depending on the design.

In determining the sterilization intensity, the microbial concentration may be considered first. In a case where the microbial concentration and the gas concentration correspond to different ranges in terms of the sterilization intensity, the sterilization intensity may be determined based on the microbial concentration. For example, in a case where the microbial concentration is greater than or equal to 1000 cfu/m³ and the gas concentration is less than or equal to 0.07 ppm, the sterilization intensity may be determined as ‘high’. In a case where the microbial concentration is less than or equal to 100 cfu/m³ and the gas concentration is greater than or equal to 0.1 ppm, the sterilization intensity may be determined as ‘low’.

The controller 500 may determine the input voltage of the fan F based on the sterilization intensity. The controller 500 may determine the input voltage of the sterilizing lamp 140 corresponding to the input voltage of the fan F. For example, in a case where the sterilization intensity is ‘low’, the input voltage of the fan F may be determined as 12 V. In addition, the input voltage of the sterilizing lamp 140 corresponding to the input voltage of the fan F may be determined as 12 V. 12 V may correspond to the default voltage of the sterilizing lamp 140. That is, the default voltage may correspond to a minimum voltage required to activate the deodorizing filter 150.

In a case where the sterilization intensity is ‘medium’, the input voltage of the fan F may be determined as 14 V. In addition, the input voltage of the sterilizing lamp 140 corresponding to the input voltage of the fan F may be determined as 14 V. In a case where the sterilization intensity is ‘high’, the input voltage of the fan F may be determined as 16 V. In addition, the input voltage of the sterilizing lamp 140 corresponding to the input voltage of the fan F may be determined as 16 V.

For example, the higher the microbial concentration and/or gas concentration in the storage compartment 20, the higher the sterilization intensity, the input voltage of the fan F, and the input voltage of the sterilizing lamp 140 may be determined. The numerical values shown in the table 700 of FIG. 7 are only examples. The numerical values for the input voltage of the fan F and the input voltage of the sterilizing lamp 140 may vary depending on the design.

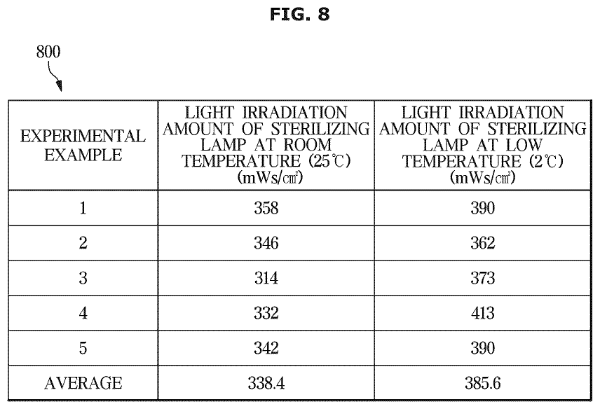

FIG. 8 is a table of experimental data showing that the light irradiation amount of a sterilizing lamp varies with temperature.

Referring to the table 800 of FIG. 8, a performance of the sterilizing lamp 140 may vary depending on an environment where the sterilizing lamp 140 is located. Table 800 of FIG. 8 shows experimental examples of comparison between the light irradiation amount of the sterilizing lamp 140 at room temperature (25° C.) and the light irradiation amount of the sterilizing lamp 140 at low temperature (2° C.). In each experimental example, other conditions (e.g., a size of the sterilizing lamp 140, a distance between the sterilizing lamp 140 and a location where the light irradiation amount is measured, and an input voltage of the sterilizing lamp 140) other than the temperature around the sterilizing lamp 140 are the same.

The light irradiation amount of the sterilizing lamp 140 was measured five times at room temperature (25° C.), and the light irradiation amount of the sterilizing lamp 140 was measured five times at low temperature (2° C.). The light irradiation amount of the sterilizing lamp 140 is shown to be larger at low temperature (2° C.) than at room temperature (25° C.). An average value of the light irradiation amount at room temperature (25° C.) is 338.4 mWs/cm², and an average value of the light irradiation amount at low temperature (2° C.) is 385.6 mWs/cm².

According to the experimental data, the sterilization performance of the sterilizing lamp 140 is higher at relatively low temperatures. The sterilization performance of the sterilizing lamp 140 may be reduced in an environment having a relatively high temperature. In other words, even in a case where the input voltage of the sterilizing lamp 140 in a low temperature environment is lower than the input voltage of the sterilizing lamp 140 in a room temperature environment, the same level of sterilization performance may be exhibited.

In addition, because the temperature of the sterilizing lamp 140 is also relatively low in a low temperature environment, there is room to increase the input voltage of the sterilizing lamp 140 until the temperature of the sterilizing lamp 140 reaches the limit temperature. For example, in a case where a default voltage (e.g., 12 V) is applied to the sterilizing lamp 140 and a temperature of the sterilizing lamp 140 is detected to be lower (e.g., 40° C.) than a limit temperature (e.g., 60° C.), the controller 500 may change the input voltage of the sterilizing lamp 140 to a maximum voltage (e.g., 16 V). An increase in the input voltage of the sterilizing lamp 140 may increase the intensity of ultraviolet light emitted from the sterilizing lamp 140, thereby improving the sterilizing effect of air passing through the sterilizing lamp 140.

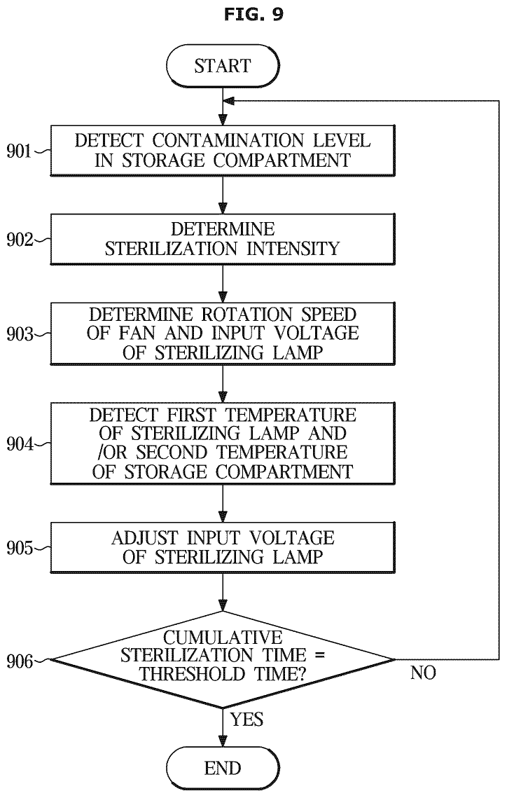

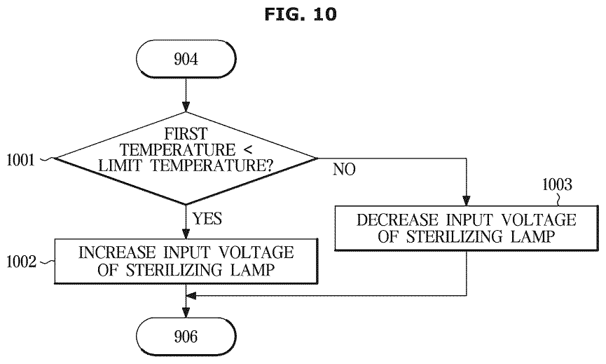

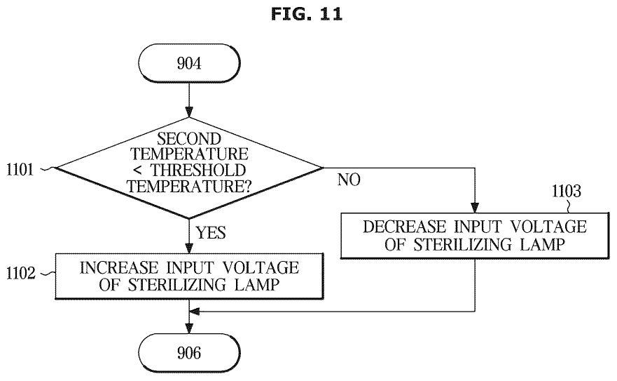

FIG. 9 is a flowchart illustrating an example method for controlling a refrigerator according to various embodiments. FIG. 10 is a flowchart illustrating an example method for adjusting an input voltage of the sterilizing lamp of FIG. 9 according to various embodiments. FIG. 11 is a flowchart illustrating an example method for adjusting an input voltage of the sterilizing lamp of FIG. 9 according to various embodiments.

Referring to FIG. 9, once a sterilization operation of the refrigerator 1 starts, the refrigerator 1 may detect a contamination level in the storage compartment 20 (901). The controller 500 of the refrigerator 1 may start the sterilization operation based on the door 30 of the storage compartment 20 being opened and then closed. In addition, the controller 500 may perform the sterilization operation at defined time intervals. The controller 500 may operate the fan F and the sterilizing lamp 140 for the sterilization operation. The controller 500 may determine the contamination level in the storage compartment 20 by receiving information about a contamination level of the storage compartment 20 from the contamination sensor 200 or based on at least one of a microbial concentration or a gas concentration detected by the contamination sensor 200.

The controller 500 may determine a sterilization intensity to perform the sterilization operation (902). The controller 500 may determine the sterilization intensity for sterilizing the inside of the storage compartment 20 based on the contamination level in the storage compartment 20. For example, the controller 500 may determine the sterilization intensity to be stronger as the microbial concentration in the storage compartment 20 is higher. The controller 500 may determine the sterilization intensity to be stronger as the gas concentration in the storage compartment 20 is higher.

The controller 500 may determine a rotation speed of the fan F and an input voltage of the sterilizing lamp 140 based on the sterilization intensity (903). The controller 500 may determine the input voltage of the sterilizing lamp 140 to correspond to the rotation speed of the fan F and/or the input voltage of the fan F. For example, the higher the sterilization intensity is determined, the higher the rotation speed of the fan F may be determined. The higher the sterilization intensity is determined, the higher the input voltage of the sterilizing lamp 140 may be determined.

As the rotation speed of the fan F increases, a flow rate of cold air passing through the sterilizing lamp 140 increases. As the flow rate of the cold air increases, the time during which the cold air is exposed to ultraviolet light emitted from the sterilizing lamp 140 becomes shorter. In a case where there is no change in the intensity of the ultraviolet light emitted from the sterilizing lamp 140 despite the increased flow rate of the cold air, a sterilizing rate may decrease. Accordingly, the input voltage of the sterilizing lamp 140 may be determined to correspond to the rotation speed of the fan F.

The refrigerator 1 may detect a first temperature of the sterilizing lamp 140 and/or a second temperature of the storage compartment 20 (904). The controller 500 of the refrigerator 1 may detect the first temperature of the sterilizing lamp 140 based on a signal transmitted from the first temperature sensor 310. The controller 500 may detect the second temperature of the storage compartment 20 based on a signal transmitted from the second temperature sensor 320.

The refrigerator 1 may adjust the input voltage of the sterilizing lamp 140 (905). In order to maximize and/or increase/improve a performance of the sterilizing lamp 140, the controller 500 may adjust the input voltage of the sterilizing lamp 140 based on the first temperature of the sterilizing lamp 140 or the second temperature of the storage compartment 20.

Referring to FIG. 10, the controller 500 of the refrigerator 1 may identify whether the first temperature of the sterilizing lamp 140 is lower than a defined limit temperature (1001). The controller 500 may increase the input voltage of the sterilizing lamp 140 (1002), based on the first temperature of the sterilizing lamp 140 being lower than the defined limit temperature. In a case where the first temperature of the sterilizing lamp 140 is lower than the defined limit temperature, there is room to increase the input voltage of the sterilizing lamp 140. By increasing the input voltage of the sterilizing lamp 140, the sterilization performance may be further improved.

The controller 500 may decrease the input voltage of the sterilizing lamp 140 (1003), based on the first temperature of the sterilizing lamp 140 reaching the defined limit temperature. In a case where the sterilizing lamp 140 continues to operate while the temperature of the sterilizing lamp 140 exceeds the limit temperature, the sterilizing lamp 140 may fail due to overheating. In a case where the temperature of the sterilizing lamp 140 reaches the limit temperature, malfunction of the sterilizing lamp 140 may be prevented and/or reduced by reducing the input voltage of the sterilizing lamp 140.

Referring to FIG. 11, the controller 500 of the refrigerator 1 may identify whether the second temperature of the storage compartment 20 is lower than a defined threshold temperature (1101). The controller 500 may increase the input voltage of the sterilizing lamp 140 (1102), based on the second temperature of the storage compartment 20 being lower than the defined threshold temperature.

The temperature of the storage compartment 20 may affect the temperature of the sterilizing lamp 140. The cold air discharged from the cold air duct 60 may pass through the sterilizing lamp 140 and then be supplied to the storage compartment 20. Accordingly, the temperature of the storage compartment 20 is related to the temperature of the sterilizing lamp 140. For example, the lower the temperature of the storage compartment 20, the lower the temperature of the sterilizing lamp 140 may be detected. In a case where the temperature of the storage compartment 20 is lower than the threshold temperature, the temperature of the sterilizing lamp 140 may be determined to be lower than the limit temperature. Accordingly, in a case where the temperature of the storage compartment 20 is lower than the threshold temperature, the controller 500 may increase the input voltage of the sterilizing lamp 140.

The controller 500 may decrease the input voltage of the sterilizing lamp 140 (1103), in a case where the second temperature of the storage compartment 20 is higher than or equal to the defined threshold temperature. In a case where the temperature of the storage compartment 20 is higher than or equal to the threshold temperature, the temperature of the sterilizing lamp 140 may be determined to be close to the limit temperature. The temperature of the sterilizing lamp 140 close to the limit temperature may indicate that a difference between the temperature of the sterilizing lamp 140 and the limit temperature is small. Accordingly, in a case where the temperature of the storage compartment 20 is higher than or equal to the threshold temperature, the input voltage of the sterilizing lamp 140 may be reduced to prevent and/or reduce a failure of the sterilizing lamp 140.

Referring back to FIG. 9, the controller 500 may determine whether a cumulative sterilization time reaches a defined threshold time (906). The controller 500 may end the sterilization operation based on the cumulative sterilization time reaching the defined threshold time. In a case where the cumulative sterilization time has not reached the defined threshold time, the controller 500 may continue to perform the sterilization operation and may periodically perform the detection of the contamination level of the storage compartment 20, the determination of the sterilization intensity, the determination of the rotation speed of the fan F, and the determination of the input voltage of the sterilizing lamp 140. The rotation speed of the fan F and the input voltage of the sterilizing lamp 140 may be changed according to a change in the contamination level of the storage compartment 20. In addition, the input voltage of the sterilizing lamp 140 may be adjusted according to the temperature of the sterilizing lamp 140 or the temperature of the storage compartment 20.

Ending the sterilization operation may include turning off the sterilizing lamp 140 or applying a defined default voltage to the sterilizing lamp 140. In a case where the refrigerator 1 includes the deodorizing lamp 160, the deodorizing filter 150 may be activated by the deodorizing lamp 160, and thus the controller 500 may stop applying voltage to the sterilizing lamp 140 and turn off the sterilizing lamp 140 based on the cumulative sterilization time reaching the threshold time. In a case where the refrigerator 1 does not include the deodorizing lamp 160, the controller 500 may apply a default voltage to the sterilizing lamp 140 to maintain an activation state of the deodorizing filter 150. For example, the sterilizing lamp 140 may be maintained to be turned on to continue a deodorization operation even in a case where the sterilization operation is completed.

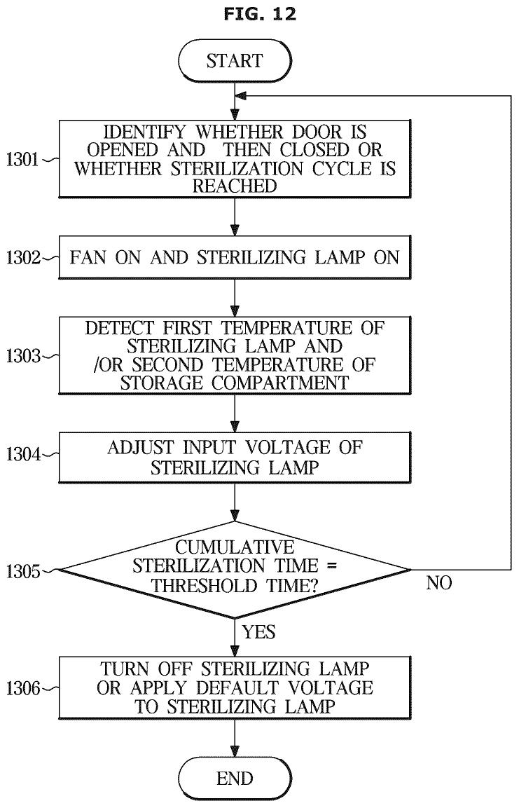

FIG. 12 is a flowchart illustrating an example method for controlling a refrigerator to adjust an input voltage of a sterilizing lamp based on a temperature of the sterilizing lamp or a temperature of a storage compartment according to various embodiments.

Referring to FIG. 12, the controller 500 of the refrigerator 1 may identify whether the door 30 of the storage compartment 20 is opened and then closed or whether a sterilization cycle is reached (1301). Each time the controller 500 detects that the door 30 of the storage compartment 20 is opened and then closed or at each defined sterilization cycle (e.g., at each defined time interval), the controller 500 may start a sterilization operation, operate the fan F (ON), and operate the sterilizing lamp 140 (ON) for the sterilization operation (1302).

In FIG. 12, the operation of determining the sterilization intensity and the operation of determining the rotation speed of the fan F and the input voltage of the sterilizing lamp 140 in response to the sterilization intensity are omitted.

The refrigerator 1 may detect a first temperature of the sterilizing lamp 140 and/or a second temperature of the storage compartment 20 (1303). The controller 500 of the refrigerator 1 may detect the first temperature of the sterilizing lamp 140 based on a signal transmitted from the first temperature sensor 310. The controller 500 may detect the second temperature of the storage compartment 20 based on a signal transmitted from the second temperature sensor 320.

The refrigerator 1 may adjust the input voltage of the sterilizing lamp 140 (1304). In order to maximize/increase/improve a performance of the sterilizing lamp 140, the controller 500 may adjust the input voltage of the sterilizing lamp 140 based on the first temperature of the sterilizing lamp 140 or the second temperature of the storage compartment 20. Operation 1304 corresponds to the above-described operation 905. Operation 1304 may include operation 1001, operation 1002, and operation 1003.

The controller 500 may determine whether a cumulative sterilization time reaches a defined threshold time (1305). The controller 500 may turn off the sterilizing lamp 140 or apply a defined default voltage to the sterilizing lamp 140 (1306), based on the cumulative sterilization time reaching the defined threshold time. The default voltage may correspond to a minimum voltage required to activate the deodorizing filter 150. In a case where the refrigerator 1 does not include the deodorizing lamp 160, the sterilizing lamp 140 may not be turned off in order to maintain the activation state of the deodorizing filter 150.

According to an example embodiment of the disclosure, a refrigerator may include: a storage compartment; a cold air duct disposed behind the storage compartment; a fan disposed in the cold air duct; a sterilizing lamp configured to emit ultraviolet light to sterilize air supplied to the storage compartment through the cold air duct; a contamination sensor configured to detect a contamination level in the storage compartment; a first temperature sensor configured to detect a first temperature of the sterilizing lamp; a second temperature sensor configured to detect a second temperature of the storage compartment; and a controller including at least one processor, comprising processing circuitry, individually and/or collectively, configured to: control the fan and the sterilizing lamp; determine a sterilization intensity based on the contamination level detected by the contamination sensor; determine a rotation speed of the fan and an input voltage of the sterilizing lamp corresponding to the rotation speed of the fan, based on the determined sterilization intensity; and adjust the input voltage of the sterilizing lamp based on the first temperature of the sterilizing lamp or the second temperature of the storage compartment.

At least one processor of the controller, individually and/or collectively, may be configured to determine the rotation speed of the fan and the input voltage of the sterilizing lamp to be higher, as the sterilization intensity is determined to be higher.

At least one processor of the controller, individually and/or collectively, may be configured to increase the input voltage of the sterilizing lamp, based on the first temperature of the sterilizing lamp being lower than a defined limit temperature.

At least one processor of the controller, individually and/or collectively, may be configured to decrease the input voltage of the sterilizing lamp, based on the first temperature of the sterilizing lamp reaching the defined limit temperature.

At least one processor of the controller, individually and/or collectively, may be configured to increase the input voltage of the sterilizing lamp, based on the second temperature of the storage compartment being lower than a defined threshold temperature.

At least one processor of the controller, individually and/or collectively, may be configured to decrease the input voltage of the sterilizing lamp, based on the second temperature of the storage compartment being greater than or equal to the defined threshold temperature or based on the first temperature of the sterilizing lamp reaching a defined limit temperature.

At least one processor of the controller, individually and/or collectively, may be configured to turn off the sterilizing lamp or apply a defined default voltage to the sterilizing lamp, based on a cumulative sterilization time reaching a defined threshold time.

At least one processor of the controller, individually and/or collectively, may be configured to determine the sterilization intensity to perform a sterilization operation, based on a door of the storage compartment being closed after being opened.

At least one processor of the controller, individually and/or collectively, may be configured to determine the sterilization intensity to perform a sterilization operation at defined time intervals.

The contamination sensor may include at least one of a microbial sensor configured to detect a microbial concentration or a gas sensor configured to detect a gas concentration. The contamination level may include at least one of the microbial concentration or the gas concentration.

According to an example embodiment of the disclosure, a method for controlling a refrigerator may include: detecting a contamination level in a storage compartment by a contamination sensor; determining a sterilization intensity based on the detected contamination level; determining a rotation speed of a fan, configured to move air to the storage compartment, and an input voltage of a sterilizing lamp corresponding to the rotation speed of the fan, based on the determined sterilization intensity, and adjusting the input voltage of the sterilizing lamp based on a first temperature of the sterilizing lamp or a second temperature of the storage compartment.

The rotation speed of the fan and the input voltage of the sterilizing lamp may be determined to be higher, as the sterilization intensity is determined to be higher.

The adjusting of the input voltage of the sterilizing lamp may include increasing the input voltage of the sterilizing lamp based on the first temperature of the sterilizing lamp being lower than a defined limit temperature.

The adjusting of the input voltage of the sterilizing lamp may include decreasing the input voltage of the sterilizing lamp based on the first temperature of the sterilizing lamp reaching the defined limit temperature.

The adjusting of the input voltage of the sterilizing lamp may include increasing the input voltage of the sterilizing lamp based on the second temperature of the storage compartment being lower than a defined threshold temperature.

The adjusting of the input voltage of the sterilizing lamp may include decreasing the input voltage of the sterilizing lamp, based on the second temperature of the storage compartment being greater than or equal to the defined threshold temperature or based on the first temperature of the sterilizing lamp reaching a defined limit temperature.

The method may further include turning off the sterilizing lamp or applying a defined default voltage to the sterilizing lamp, based on a cumulative sterilization time reaching a defined threshold time.

The determining of the sterilization intensity may be performed based on a door of the storage compartment being closed after being opened.

The determining of the sterilization intensity may be performed at defined time intervals.

The determining of the sterilization intensity may be based on at least one of a microbial concentration detected by a microbial sensor or a gas concentration detected by a gas sensor.

According to the disclosure, the refrigerator and the method for controlling the same may control not only a rotation speed of a fan for supplying cold air but also the light irradiation amount from a sterilizing lamp depending on a contamination level of a storage compartment, and thus even in a case where a flow rate of cold air increases due to an increase in the rotation speed of the fan, a sterilization rate may be prevented/reduced from decreasing and a sterilization time may be shortened.

In addition, the refrigerator and the method for controlling the same may maximize/increase/improve sterilization performance by controlling an input voltage of a sterilizing lamp according to a temperature of the sterilizing lamp or a temperature of a storage compartment.

The disclosed embodiments may be implemented in the form of a recording medium that stores instructions executable by a computer. The instructions may be stored in the form of program codes, and when executed by a processor, the instructions may create a program module to perform operations of the disclosed embodiments.

The machine-readable recording medium may be provided in the form of a non-transitory storage medium, wherein the ‘non-transitory storage medium’ is a storage medium that is tangible device, and may not include a signal (e.g., an electromagnetic wave), but this term does not differentiate between where data is semi-permanently stored in the storage medium and where the data is temporarily stored in the storage medium. For example, a ‘non-transitory storage medium’ may include a buffer in which data is temporarily stored.

According to an embodiment, the method according to the various embodiments disclosed herein may be provided in a computer program product. The computer program product may be traded between a seller and a buyer as a product. The computer program product may be distributed in the form of a machine-readable storage medium (e.g., compact disc read only memory (CD-ROM)), or may be distributed (e.g., download or upload) through an application store (e.g., Play Store™) online or directly between two user devices (e.g., smartphones). In the case of online distribution, at least a portion of the computer program product (e.g., downloadable app) may be stored at least semi-permanently or may be temporarily generated in a storage medium, such as a memory of a server of a manufacturer, a server of an application store, or a relay server.

While the disclosure has been described with reference to various example embodiments and the accompanying drawings, one skilled in the art will appreciate that various modifications may be easily made without departing from the technical spirit or essential features of the disclosure, including the appended claims and their equivalents. Therefore, the foregoing embodiments should be regarded as illustrative rather than limiting in all aspects. It will also be understood that any of the embodiment(s) described herein may be used in conjunction with any other embodiment(s) described herein.

What is claimed is:

1. A refrigerator, comprising:
a storage compartment;
a cold air duct disposed behind the storage compartment;
a fan disposed in the cold air duct;
a sterilizing lamp configured to emit ultraviolet light to sterilize air supplied to the storage compartment through the cold air duct;
a contamination sensor configured to detect a contamination level in the storage compartment;
a first temperature sensor configured to detect a first temperature of the sterilizing lamp;
a second temperature sensor configured to detect a second temperature of the storage compartment; and
a controller including at least one processor, comprising processing circuitry, individually and/or collectively configured to: control the fan and the sterilizing lamp,
determine a sterilization intensity based on the contamination level detected by the contamination sensor,
determine a rotation speed of the fan and an input voltage of the sterilizing lamp corresponding to the rotation speed of the fan, based on the determined sterilization intensity, and
adjust the input voltage of the sterilizing lamp based on the first temperature of the sterilizing lamp or the second temperature of the storage compartment.

2. The refrigerator of claim 1, wherein at least one processor of the controller, individually and/or collectively, is configured to determine the rotation speed of the fan and the input voltage of the sterilizing lamp to be higher, as the sterilization intensity is determined to be higher.

3. The refrigerator of claim 1, wherein at least one processor of the controller, individually and/or collectively, is configured to increase the input voltage of the sterilizing lamp, based on the first temperature of the sterilizing lamp being lower than a defined limit temperature.

4. The refrigerator of claim 3, wherein at least one processor of the controller, individually and/or collectively, is configured to decrease the input voltage of the sterilizing lamp, based on the first temperature of the sterilizing lamp reaching the defined limit temperature.

5. The refrigerator of claim 1, wherein at least one processor of the controller, individually and/or collectively, is configured to increase the input voltage of the sterilizing lamp, based on the second temperature of the storage compartment being lower than a defined threshold temperature.

6. The refrigerator of claim 5, wherein at least one processor of the controller, individually and/or collectively, is configured to decrease the input voltage of the sterilizing lamp, based on the second temperature of the storage compartment being greater than or equal to the defined threshold temperature or based on the first temperature of the sterilizing lamp reaching a defined limit temperature.

7. The refrigerator of claim 1, wherein at least one processor of the controller, individually and/or collectively, is configured to turn off the sterilizing lamp or apply a defined default voltage to the sterilizing lamp, based on a cumulative sterilization time reaching a defined threshold time.

8. The refrigerator of claim 1, wherein at least one processor of the controller, individually and/or collectively, is configured to determine the sterilization intensity to perform a sterilization operation, based on a door of the storage compartment being closed after being opened.

9. The refrigerator of claim 1, wherein at least one processor of the controller, individually and/or collectively, is configured to determine the sterilization intensity to perform a sterilization operation at defined time intervals.

10. The refrigerator of claim 1, wherein the contamination sensor includes at least one of a microbial sensor configured to detect a microbial concentration or a gas sensor configured to detect a gas concentration, and
the contamination level includes at least one of the microbial concentration or the gas concentration.

11. A method for controlling a refrigerator, comprising:
detecting a contamination level in a storage compartment by a contamination sensor;
determining a sterilization intensity based on the detected contamination level;
determining a rotation speed of a fan, configured to move air to the storage compartment, and an input voltage of a sterilizing lamp corresponding to the rotation speed of the fan, based on the determined sterilization intensity, and
adjusting the input voltage of the sterilizing lamp based on a first temperature of the sterilizing lamp or a second temperature of the storage compartment.

12. The method of claim 11, wherein the rotation speed of the fan and the input voltage of the sterilizing lamp are determined to be higher, as the sterilization intensity is determined to be higher.

13. The method of claim 11, wherein the adjusting of the input voltage of the sterilizing lamp comprises increasing the input voltage of the sterilizing lamp based on the first temperature of the sterilizing lamp being lower than a defined limit temperature.

14. The method of claim 13, wherein the adjusting of the input voltage of the sterilizing lamp comprises decreasing the input voltage of the sterilizing lamp based on the first temperature of the sterilizing lamp reaching the defined limit temperature.

15. The method of claim 11, wherein the adjusting of the input voltage of the sterilizing lamp comprises increasing the input voltage of the sterilizing lamp based on the second temperature of the storage compartment being lower than a defined threshold temperature.
